(19) United States
(12) Patent Application Publication
(10) Pub. No.: US 2009/0024176 A1
(43) Pub. Date: Jan. 22, 2009

(54) METHODS AND DEVICES FOR PRODUCING RESPIRATORY SINUS ARRHYTHMIA

(76) Inventors: Joonkyoo Anthony Yun, Palo Alto, CA (US); Anthony Gerber, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/174,379

(22) Filed: Jul. 16, 2008

Related U.S. Application Data
(60) Provisional application No. 60/961,008, filed on Jul. 17, 2007.

(51) Int. Cl. A61N 1/365

(52) U.S. Cl. 607/20; 607/17

(57) ABSTRACT

Methods and devices for producing respiratory sinus arrhythmia in a subject are provided. Aspects of the invention include electrically stimulating or sensing motion of the diaphragm, and electrically stimulating the heart in a subject in a manner effective to produce respiratory sinus arrhythmia. The methods and devices find use in a variety of applications, e.g. in the treatment of subjects suffering from heart failure conditions.

### CROSS-REFERENCE TO RELATED APPLICATIONS

Pursuant to 35 U.S.C. § 119(e), this application claims priority to the filing date of U.S. Provisional Patent Application Ser. No. 60/961,008 filed Jul. 17, 2007; the disclosure of which is herein incorporated by reference.

### INTRODUCTION

Respiratory sinus arrhythmia (RSA) is a well known phenomenon in healthy individuals in which the heart rate varies in synchrony with respiration. During RSA, the interval between heartbeats (R-R interval on an EKG) is shortened during inspiration and lengthened during expiration, which results in improved coordination between the timing of alveolar ventilation and perfusion in each respiratory cycle. By decreasing the number of heartbeats during expiration, and increasing the number of heartbeats during inspiration, the efficiency of gas exchange at the level of the lung is improved.

RSA or heart rate variability (HRV) in synchrony with respiration is a physiologic phenomenon which has been used as an index of cardiac vagal function. In patients with heart failure, heart rate variability, of which RSA is the primary component, is markedly decreased. A reduction in HRV is associated with increased mortality, and has been shown to be a better predictor of death due to progressive heart failure than other traditional clinical measures such as left ventricular size or history of non-sustained ventricular tachycardia.

### SUMMARY

Methods and devices for producing respiratory sinus arrhythmia in a subject are provided. Aspects of the invention include electrically stimulating or sensing motion of the diaphragm, and electrically stimulating the heart in a subject in a manner effective to produce respiratory sinus arrhythmia. The methods and devices find use in a variety of applications, e.g. in the treatment of subjects suffering from heart failure conditions.

### BRIEF DESCRIPTION OF THE DRAWINGS

FIG. 1 shows a diagram of an implantable diaphragm stimulation and sensing device, and an implantable cardiac pulse generator with wireless communication in accordance with an embodiment of the present invention.

FIG. 2 shows a diagram of an implantable diaphragm stimulation and sensing device, and an implantable cardiac pulse generator with a hardwired connection in accordance with an embodiment of the present invention.

FIG. 3 shows a block diagram of an implantable pulse generator in accordance with an embodiment of the present invention.

### DETAILED DESCRIPTION

Methods and devices for producing respiratory sinus arrhythmia in a subject are provided. Aspects of the invention include electrically stimulating or sensing motion of the diaphragm, and electrically stimulating the heart in a subject in a manner effective to produce respiratory sinus arrhythmia. The methods and devices find use in a variety of applications, e.g. in the treatment of subjects suffering from heart failure conditions.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing embodiments of the invention, aspects of the methods will be described first, followed by a review of aspects of devices for use in the subject methods, embodiments of applications in which the methods and devices find use, as well as kits for performing methods of the invention.

### Methods

Aspects of the invention include methods of electrically stimulating or sensing movement of diaphragm tissue and cardiac tissue in a subject in a manner effective to produce respiratory sinus arrhythmia (RSA). During RSA, the interval between heartbeats (R-R interval on an EKG) is shortened during inspiration and lengthened during expiration, which results in improved coordination between the timing of alveolar ventilation and perfusion in each respiratory cycle. By decreasing the number of heartbeats during expiration, and increasing the number of heartbeats during inspiration, the efficiency of gas exchange at the level of the lung is improved. In certain embodiments, RSA produced according to the invention is characterized by decreasing the number of heartbeats during expiration (as compared to a control non-RSA state) by 5% or more, such as by 20% or more and including by 50% or more. In certain embodiments, RSA produced according to the invention is characterized by increasing the number of heartbeats during inspiration (as compared to a control non-RSA state) by 5% or more, such as by 20% or more and including by 50% or more.

In certain embodiments, an implantable cardiac pulse generator in a subject is coupled to an implantable diaphragm stimulator, and regulated so that the cardiac pacing rate is adjusted in synchrony with the respiration of the patient to produce respiratory sinus arrhythmia that is substantially the same as, if not identical, to that seen in healthy patients. Both the diaphragmatic stimulation and the cardiac pacing can be programmed using a “respiratory parameter”. By “respiratory parameter” is meant a parameter that reflects respiratory function, which can be used to modify the cardiac pacing rate in order to recreate respiratory sinus arrhythmia. The “respiratory parameter” can be derived from data that is sensed, or it can be a respiratory rate chosen in order to implement therapy. The “respiratory parameter” can be derived from a baseline respiratory rate sensed for a subject; determined from measurement of subject's metabolism or activity level; determined by sensing phrenic nerve activity; determined from electrical activity or detection of diaphragm movement; determined by measuring respiratory air flow (e.g. a peumotachometer); determined by a measure of pressure (e.g. an intrathoracic pressure sensor); or determined by measuring impedance (e.g. transthoracic impedance). The “respiratory parameter” can also be chosen in order to implement therapy, e.g. to improve O₂ saturation in heart failure, treat hypertension, or treat breathing disorders, etc.

In certain embodiments, the implantable diaphragmatic device is used to stimulate diaphragmatic contractions, and in some embodiments, the implantable diaphragmatic device is used to sense diaphragmatic contractions. In certain embodiments, a respiratory parameter can be derived from a respiratory flow measuring device, such as a pneumotachometer. In certain embodiments, the respiratory parameter can be derived from other measurements (e.g. an intrathoracic pressure sensor, or measures of transthoracic impedance). In some embodiments, the method of restoring respiratory sinus arrhythmia is used while the subject is sleeping. The subject methods and devices find use in a variety of therapeutic applications, including treatment for heart failure patients and patients with breathing disorders.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

### Implantable Diaphragmatic Device

Aspects of the invention include methods of electrically stimulating diaphragm tissue and cardiac tissue in a subject. An implantable device for sensing diaphragm motion and/or delivering electrical stimulation waveforms to the diaphragm through one or more electrodes can be used, as described in patent publications 2005/0085865, 2005/0085867, 2005/0085868, the disclosures of which are herein incorporated by reference. The identification of the respiratory rate, inspiration cycle, exhalation cycle rest period, and tidal volume may be accomplished by sensing the respiration waveform, e.g., with a pneumotachometer, movement detector or using EMG. An example of such determination is described in U.S. Application 2005/0085865 incorporated herein by reference. Various methods and devices that may be used to map ideal electrode placement for a desired result or to optimize stimulation to achieve such a result are described in related U.S. Application 2005/0085869 incorporated herein by reference. In the subject methods, a programmable implantable diaphragm stimulation device can provide stimulation waveforms that adjust minute ventilation, and/or manipulate subject blood gases. The implantable diaphragm stimulation device can be programmed by a programmer that can be linked to a flow sensor that measures the natural respiration or stimulated respiration of a subject. Normal breathing of the subject in an awake or sleeping state can be observed to establish a baseline reference minute ventilation, and minute ventilation can be increased or decreased based on input of a respiratory parameter (e.g. a measured or desired baseline respiratory rate). Several parameters can be adjusted in order to change the minute ventilation, including but not limited to respiratory rate, tidal volume, inspiration duration, expiration duration, flow morphology, flow rate, slope of the inspiration curve, and diaphragm-created or intrathoracic pressure gradients. The implantable diaphragm stimulation device can be linked to an implantable pulse generator, which dynamically responds to instantaneous respiration and adjusts its pacing rate accordingly. In addition to direct stimulation of the diaphragm, the electrical stimulation of the diaphragm can occur by stimulating the phrenic nerve to control breathing.

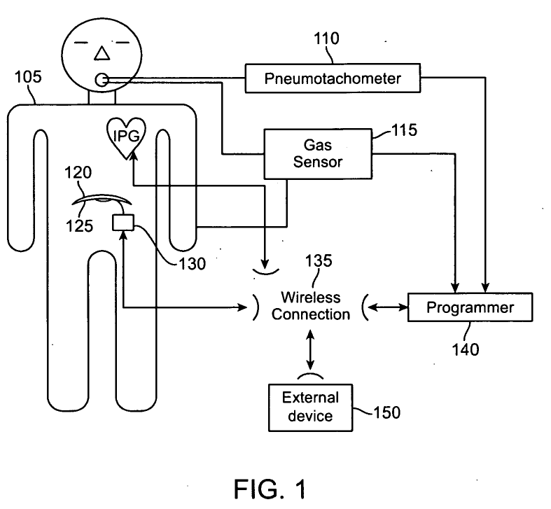

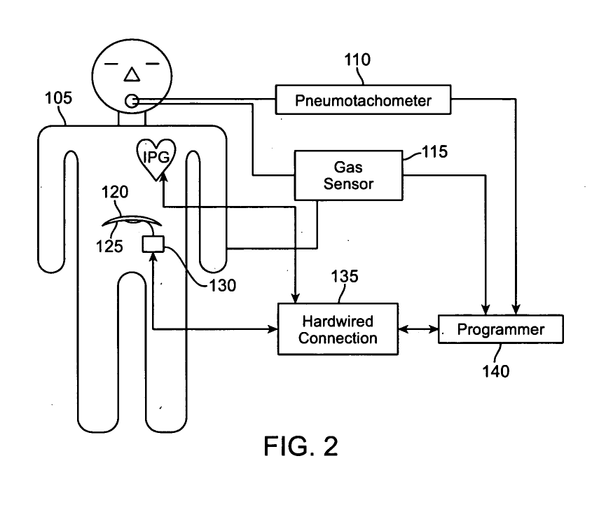

FIGS. 1 and 2 show a diagram of a system 100 for diaphragm stimulation in accordance with the invention. A subject 105 is implanted with a programmable stimulation and sensing device 130 that is coupled to one or more electrodes 125 in contact with the diaphragm 120. The one or more electrodes 125 may include both sensing and stimulation electrodes. A flow measuring device 110 (e.g., a pneumotachometer) is used to measure the respiratory flow characteristics (e.g., tidal volume, inspiration duration, and respiratory rate) of the subject 105.

A programmer 140 is coupled to the flow measuring device 110. The programmer 140 is also coupled to the implanted programmable diaphragm stimulating and sensing device 130 and to the implantable pulse generator by either a wireless connection (FIG. 1) or a hardwired connection (FIG. 2). An optional sensor 115 may also be coupled to the subject 105 and to the programmer for collecting respiratory and/or blood gas composition data (e.g., pulse oximetry or exhaled gas composition). The programmable diaphragm stimulating device 130 is implanted subcutaneously within the patient, for example in the chest region on top of the pectoral muscle.

According to one embodiment, a stimulator (130) includes an implantable controller coupled through leads to electrodes to be implanted on the diaphragm in the vicinity of the phrenic nerve branches. The electrodes may sense either nerve activity or EMG signals of the diaphragm. The stimulator may further include a pulse generator configured to deliver stimulating pulses, either to the same electrodes used for sensing or to additional stimulation electrodes. The stimulation electrodes may also be placed adjacent to the phrenic nerve at some point along its length to provide stimulation pulse to the nerves, which in turn innervate the diaphragm muscle causing contractions and resulting respiration. Alternatively the electrodes may be placed on the phrenic nerve for both sensing and stimulation.

The implantable diaphragmatic device can be used for sensing diaphragmatic motion or electrical activity, which can be used to for sensing of a respiratory parameter. In some embodiments, the respiratory parameter can be used to adjust the cardiac pacing rate. Diaphragmatic activity can also be recorded and communicated to an external device to provide information to a subject and/or provider for further treatment or diagnosis. In some embodiments movement detectors, such as strain gauges or accelerometers, are included with the electrode assemblies. The movement detectors detect movement of the diaphragm and thus the respiratory effort exerted by the diaphragm. The movement detectors can sense mechanical movement and deliver a corresponding electrical signal to a programmable diaphragm stimulating and sensing device 130 where the information is processed by a programmer 140. The movement may be used to qualify the electrical phrenic nerve or EMG signal sensed by the device to confirm inspiration or exhalation is occurring, e.g., by matching mechanical and electrical activities of the diaphragm.

The implantable diaphragmatic device can be used to provide stimulation of the diaphragm that inhibits central respiratory drive for a sufficient duration so that therapeutic stimulation and breathing control may be applied. The therapeutic stimulation breathing is configured to provide a therapeutic benefit at the same time that it acts to inhibit central respiratory drive. In one embodiment, the stimulation intensity, duration and respiratory rate are manipulated to inhibit respiratory drive while providing desired stimulation to the diaphragm. For example, at a given respiratory rate and tidal volume during diaphragm stimulation, extending the inspiration or expiration duration (among other things, by increasing stimulation duration and decreasing intensity) effectively shortens the resting period compared to spontaneous breathing and decreases the likelihood of a spontaneous breath between stimulations. Breathing disorders such as apnea or hypoventilation may be treated by electrically stimulating the diaphragm tissue or phrenic nerve in response to a sensed respiratory parameter or characteristic.

This therapy mode can be maintained for a programmable amount of time, e.g., for one or more intervals of time during the night or during the day. After the breathing therapy mode, breathing is normalized to allow PCO₂ to slowly increase so spontaneous breathing can be restored. This may be accomplished by returning respiratory rate back to normal and maintaining normal tidal volume to increase PCO2 and thereby encourage the return of intrinsic breathing and respiratory drive. If after the stimulator stimulates breathing at a normal rate for a period of time and spontaneous breathing has not returned, the patient is weaned from the stimulator by further decreasing the respiratory rate and therefore minute ventilation. This will allow intrinsic breathing and respiratory drive to return by allowing an increase in PCO2.

### Implantable Pulse Generator

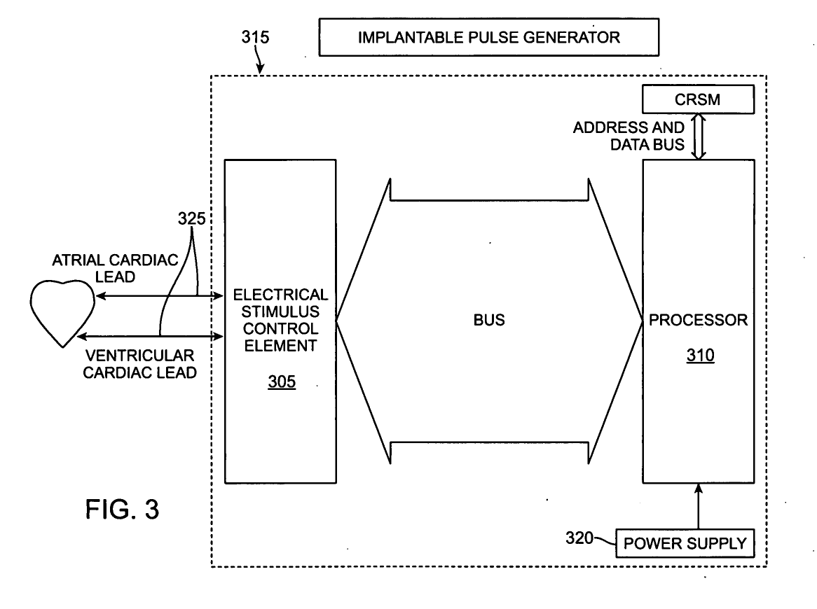

Embodiments of the invention further include using implantable pulse generators. The implantable pulse generators of the invention provide conventional pacing functions and are also modified to provide phasic, respiration modulated pacing (FIG. 3). An implantable pulse generator constructed in accordance with this invention includes an electrical stimulus control element (305), which generates pacing pulses in response to commands from a processor (310). The processor can first establish a baseline pacing rate, which can be based on metabolic demand using standard rate responsive pacing (i.e. sensing activity level of the patient, and adjusting the base pacing rate accordingly). The baseline pacing rate is then adjusted in accordance with a respiratory parameter, in a manner sufficient to produce respiratory sinus arrhythmia, e.g., as seen in a healthy subject.

Implantable pulse generators may include: a housing (315) which includes a power source (320) and an electrical stimulus control element (305); one or more vascular leads (325), e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator so that it dynamically responds to a respiratory parameter, and adjusts its pacing rate accordingly in a manner which accurately restores respiratory sinus arrhythmia in a subject. The processor 310 receives status and/or control inputs from the electrode sensors 325. Using the control element 305, it performs various operations, including arrhythmia detection, and produces outputs, such as the atrial pace control and ventricular pacing control, which determine the type of pacing that is to take place. The rate of the atrial and/or ventricle pacing is adjusted by the control element 305 not only to conform to the metabolic demand of the patient but also in accordance with the respiration of the patient.

In some embodiments, the electrical stimulus control element 305 can include an impedance measurement circuit for measuring a physiological parameter indicative of the patient's metabolic demand, which can be used to derive a respiratory parameter. To obtain an impedance measurement, the processor 310 can send a signal to activate an impedance measurement circuit. The impedance measurement circuit can then apply a current to the ventricular cardiac lead 325 to measure the voltage resulting from the applied current. The current and voltage signals define an impedance that is representative of the subject's metabolic demand, and more particularly, of the instantaneous minute volume. The instantaneous minute volume is then filtered and further modified by subtracting from it a long term average value. The resulting minute volume parameter can be used to derive a respiratory parameter.

The electrical stimulus control element 305 can further include a respiration detector which detects the respiration function of the patient. In one embodiment, the respiration function can be determined from the impedance measurements taken by an impedance measurement circuit as described above. The respiration detector may use other signals to detect respiration, as described in U.S. Pat. Nos. 6,454,719 and 5,964,788 incorporated herein by reference.

### Respiratory Parameter

The processor of the implantable pulse generator 310 can receive input such as a respiratory parameter. The respiratory parameter reflects respiratory function, which can be used to modify the cardiac pacing rate in order to produce respiratory sinus arrhythmia. The respiratory parameter can be derived from data that is sensed, or it can be a respiratory rate chosen in order to implement therapy. The respiratory parameter can be derived from a baseline respiratory rate observed for a subject, or obtained from measurement of a subject's metabolism or activity level. The respiratory parameter can be determined from electrical activity of the phrenic nerve, or electrical activity of the diaphragm, such as from direct measurement of an EMG of the diaphragm, either during active electrical stimulation or passive sensing of diaphragm contractions. The respiratory parameter can be determined from sensing of the motion of the diaphragm, e.g. using a strain gauge or accelerometer. In some embodiments the respiratory parameter can be determined from sensing an air flow with a flow measuring device (e.g. a pneumotachometer). In other embodiments, the respiratory parameter can be determined by measuring pressure (e.g. an intrathoracic pressure sensor), or determined by measuring impedance (e.g. transthoracic impedance). The “respiratory parameter” can also be chosen in order to implement therapy, e.g. to improve O₂ saturation in heart failure, treat hypertension, or treat breathing disorders, etc.

### Stimulate Diaphragm, Modify Cardiac Pacing Rate

In certain embodiments of the invention, the implantable diaphragm stimulator is programmed to regulate the respiration of the subject. The system shown in FIGS. 1 and 2 may be used to observe the natural normal, or intrinsic respiration of a subject in either the waking state or the sleeping state. When the flow measuring device 110 is a pneumotachometer, the subject will more likely be in the waking state. In some embodiments diaphragm stimulation is accomplished primarily at night, or while the subject is sleeping. The programmed respiratory rate can be based on a respiratory parameter determined from the baseline metabolic demand of the patient (e.g. minute ventilation, activity level) it can be a respiratory parameter chosen to manipulate respiratory blood gases, or to manipulate respiratory rate (e.g. for treatment of apnea). In certain embodiments, an implantable cardiac pulse generator in a subject is coupled to the implantable diaphragm stimulator, and regulated so that the cardiac pacing rate is adjusted in synchrony with the respiration of the patient to recreate respiratory sinus arrhythmia.

The respiratory parameter can provide information about the amplitude and timing of patient respiration, which can be used to develop rate modulation signals for controlling delivery of stimulus pulses to the implantable pulse generator. In one embodiment, the rate modulated stimulus signals are delivered to the patient's heart in the manner of conventional cardiac pacing, with an increased rate during inspiration. In some embodiments, the pacing rate may be decreased during expiration as well. The system is flexible and can provide for phasic rate control which continually looks at beat-to-beat cardiac variations and adjusts phasic rate modulation toward maintaining more uniform power output. For patients with a relatively high sinus rate, e.g., heart failure patients, the implanted pacemaker is suitably programmed only to increase heart rate during the inspiration phase; such pacing might be followed spontaneously by a compensatory decrease in sinus rhythm. In pacemaker patients with a very low sinus rhythm, the mean pacing rate is suitably set high, so that the heart can follow up and down changes in rate during inspiration and expiration, respectively. In some embodiments, an alternative to direct pacing of the heart is to stimulate the vagus nerve (or only the cardiac branch of the vagus nerve) or the sympathetic nerve system to control the beat rate of the subject's heart. This causes a short duration change of heart rate, and should be timed for delivery during inspiration or expiration. Any combination of direct cardiac pacing and vagus nerve or sympathetic nerve stimulation may be employed for a given patient. Further, the system can be programmed to be active only during rest, i.e., only if the body sensor and activity sensor outputs are below predetermined references. Further details of the method of respiration-modulated pacing are described in U.S. Pat. No. 6,141,590, incorporated herein by reference.

The system shown in FIGS. 1 and 2 may be used to determine a respiratory parameter based on a minute ventilation baseline reference value for a subject. The device 130 may be programmed to provide a waveform stimulus to adjust the minute ventilation of the subject about the baseline reference value:

Minute ventilation=tidal volume×respiration rate for one minute

Minute ventilation may be determined measuring tidal volume over time, or an instantaneous value. The minute ventilation of the subject generally increases or decreases with an increase or decrease in energy (e.g., in frequency, current, pulse width, or amplitude) applied to the diaphragm. The energy applied by the device 130 may be adjusted by selection of the amplitude, frequency, pulse width, and duration of the series of pulses or stimulus waveform applied by the device 130.

In one embodiment, the device may be programmed to produce stimulation waveforms that vary the combination of respiratory rate and tidal volume which result in an increase or decrease of minute ventilation from a reference level. Increasing minute ventilation generally increases the partial pressure of O₂ compared to a reference minute ventilation. Decreasing minute ventilation generally increases the partial pressure of CO₂ compared to a reference minute ventilation. Accordingly, the invention provides a method and device that manipulates blood gas concentration.

In another embodiment, the invention provides stimulation waveforms that are directed to manipulation of patient blood gases, e.g. SaO₂ and PCO₂. In order to achieve manipulation of blood gas concentration, in one embodiment minute ventilation is increased or decreased with respect to a baseline minute ventilation. This may be done by manipulation of one or more parameters affecting minute ventilation. Some of the parameters may include, for example, tidal volume, respiration rate, flow morphology, flow rate, inspiration duration, slope of the inspiration curve, and diaphragm-created or intrathoracic pressure gradients. The implantable device may be programmed by a programmer that is coupled to a flow sensor that measures the natural respiration and stimulation respiration of a subject. Normal breathing of a subject is observed to establish a baseline reference minute ventilation, and the device is programmed to produce stimulation waveforms that may provide either a decrease or an increase in the subject's minute ventilation. Further details of these methods are described in U.S. patent publications 2005/0085865, 2005/0085867, 2005/0085868, the disclosures of which are herein incorporated by reference.

In one embodiment of the invention the reference minute ventilation of a patient is obtained by observing normal breathing of a patient in an awake state, and increased and decreased minute ventilation are obtained by interacting with the patient. In another embodiment the reference minute ventilation of a patient is obtained by observing the patient in the sleeping state, and increased and decreased minute ventilation are obtained by applying a predetermined multiplier. The multiplier is used to scale by one or more of the scaling constants S1, S2, S3. Scaling constants S1 and S2 are indicative of the patient's age and physical fitness. Each of these scaling factors tend to affect the respiratory sinus arrhythmia. More specifically, RSA decreases with age, increases with improved fitness, and decreases during exercise.

### Sense Respiration, Modify Cardiac Pacing Rate

In certain embodiments, the implantable diaphragmatic device is used to sense diaphragmatic contractions in order to derive a respiratory parameter that can be used to adjust the cardiac pacing rate. In certain embodiments, the same electrodes used for diaphragm stimulation can be used to sense respiration by sensing diaphragm motion, and in certain embodiments, separate sensing electrodes on the implantable diaphragm stimulator can be used to sense diaphragm motion, or diaphragm EMG. In certain embodiments, motion of the diaphragm can be detected by a motion sensing device (e.g. a strain gauge, or an accelerometer) on the implantable diaphragm stimulator. In certain embodiments, motion of the diaphragm can be detected by sensing activity of the phrenic nerve. In certain embodiments, the respiratory parameter can be derived from other devices, such as a flow measuring device (e.g. a pneumotachometer). In certain embodiments, the respiratory parameter can be derived from an intrathoracic pressure sensor (e.g. an intrathoracic pressure sensor); or determined by measuring impedance (e.g. transthoracic impedance), as described in U.S. patent publications 2005/0085865, 2005/0085867, 2005/0085868, and U.S. Pat. Nos. 6,141,590, 5,964,788, and 6,454,719 incorporated herein by reference.

In one embodiment, a respiratory parameter is derived which corresponds to respiratory effort of the diaphragm, and electrical stimulating pulses are delivered to the heart based on the sensed information.

### Method of Diagnosis and Treatment

The methods of the subject invention can be used for the diagnosis and treatment of a heart failure condition, such as an early heart failure condition. In one embodiment, the methods of the subject invention can be used to provide a cardiac treatment system which monitors a patient's cardiac condition over a predetermined time period and, based on the patient's breathing pattern, indicates whether the patient's condition has improved, worsened or remaining unchanged. It is known that there is a correlation between the respiration rate variability of a cardiac patient and the progression of the patient's heart condition. Heart failure patients typically have a characteristic breathing pattern which is dominated by a low-frequency variation as compared to healthy persons. More specifically, heart failure patients have a periodic breathing pattern characterized by sequential deep and shallow breaths as compared to the breathing pattern of a healthy person. The variability of the respiration parameter is used to generate a signal indicative of the current heart failure status of the patient, and more particularly whether the patient's condition has improved, worsened, or remained unchanged over a predetermined time period. These methods are further described in U.S. Pat. No. 6,454,719, incorporated herein by reference.

Hypoventilation may be detected where the respiratory rate or frequency falls below a programmed rate. Hyperventilation may be detected when the respiratory rate or frequency is above a programmed rate. Complete apnea or central apnea is defined as a condition where there is no effective EMG signal or phrenic nerve signal, i.e. where there is no effective or significant physiological response. Frequently, a hyperventilation episode is followed by loss of diaphragm EMG or phrenic nerve activity. The device may be programmed to first detect the hyperventilation and wait for a preprogrammed time to be considered apnea. For example, the time may be set to 10-20 seconds of lost EMG after a hyperventilation episode to detect complete apnea. Partial apnea or obstructive sleep apnea is defined to be present when the EMG or phrenic nerve activity is attenuated and may be detected when the amplitude drops below a programmed amount. For example such amount may be based on the EMG or phrenic nerve amplitude dropping a percentage, e.g. 50% of the baseline EMG amplitude. Also the phase of the respiratory cycles in partial apnea may be determined or compared to an in phase cycle. An out of phase or arrhythmic cycle may also be used to detect partial apnea.

A number of different parameters may be programmed into the processor to determine if certain breathing disorders are present, and when and how to stimulate respiration, and when to stop or modify stimulation. Phrenic nerve or EMG activity sensed may include, for example, amplitude, frequency, and waveform to determine central respiratory efforts, the absence, a decrease in amplitude, abnormalities in frequency and/or amplitude, or waveform morphology of which may indicate the onset of apnea, hyperventilation, or hypoventilation. The nerve activity may be compared to predetermined activity levels or patient historical activity. Similarly, diaphragm EMG amplitude, frequency, waveform morphology and history may be used to determine apnea, hyperventilation and hypoventilation. For example, the nerve activity at the onset of sleep or after a given time in a reclining position, may be used as a baseline for comparison.

Accelerometer information may be used to determine information regarding patient's physical activity, e.g., to match/compare to the respiratory patterns and activities and collect data on related patient activities, respiratory activities, and create or adjust a treatment plan based thereon, (e.g., modification of diuretics or ACE inhibitors). Accelerometer sensors may also be used to determine information regarding movement pattern of the diaphragm muscles, intercostal muscles, and rib movement and thus determine overall respiratory activity and patterns.

Stimulation of respiration may be initiated when “no” or “attenuated” respiratory activity has been present or detected for a time period (when apnea is detected). The time period may be pre-programmed for a specific patient by the physician, as otherwise preset, or as determined a program in the treatment device. The device may be programmable for other breathing disorders, allowing slow or fast inspiration and visa versa allowing slow or fast expiration. For example, based on programmed parameters of the activity sensor, for patients suffering from hypoventilation, the inspiration rate may be increased or decreased based on the level of activity.

The information that may be downloaded for pulmonary edema management (e.g., of hyperventilation rate and frequency of occurrence) may include the detections rate, detection amplitude, ventilation waveform morphology including slopes and surface of inspiration waveform, slopes and surface area of exhalation waveform, recorded respiratory waveform information in conjunction with activity and position sensors information. The information that may be downloaded for sleep apnea treatment may include, e.g., detection rate, detection amplitude, pacing therapy amplitude, pacing pulse width, pacing frequency or other stimulation waveform morphology. This information may be used to calibrate device detection and therapy parameters.

A provider may use the information in developing an optimum treatment plan for the patient including drug titrations for diuretic management as well as if patient is in need of urgent attention leading to hospitalization, which is a frequent occurrence with heart failure patients dealing with pulmonary edema. The patient compliance information may also be used for understanding the drug regimen effectiveness if patient complies or educate the patient when there is lack of compliance with the therapy plan.

The system EMG memory is programmable to pre-trigger and post trigger lengths of storage for sleep apnea episodes. The pre-trigger events are the waveform signals and other sensed information observed transitioning to an event. Post-trigger events are the waveforms and other sensed information observed after an event and/or after treatment of an event, to observe how the device operated. Post-trigger recordings can confirm if the episode was successfully treated. The pre-trigger and post-trigger time periods can be preprogrammed into the programmable stimulation and sensing device 130.

The programmable stimulation and sensing device 130 can also include an accelerometer configured to sense acceleration and movement of the patient and to provide a digital signal corresponding to the sensed movement to the processor. In addition, an accelerometer can be positioned within the programmable stimulation and sensing device 130. The accelerometer measures the activity levels of the patient and provides the signal to the programmer 140 for use in further analysis. Using an accelerometer in the implanted device indicates the activity level of the patient in conjunction with breathing rate. The accelerometer senses activity threshold as at rest, low medium or high depending on the programmed threshold value for a specific patient. Using the activity (accelerometer) sensor value and respiratory information, the health of the respiratory system may be evaluated and monitored. For example, if a patient's respiratory rate increases with an increase in activity and decreases with a decrease in activity, within a normal range, the patient's system will be considered functioning normally. If the patient's respiratory rate is out of range or too high while the activity sensor indicates at rest or low, then the patient may be suffering from pulmonary edema. Using this monitor, the effect of drug titrations, e.g., diuretic dosages, on a patient with pulmonary edema can be monitored. If the pulmonary edema patient's respiration is brought more towards a normal range with a drug dose, then the drug treatment would be maintained. If the drug treatment did not effect breathing sufficiently then the drug dosage may be increased. Accordingly, the drug dosage may vary with detected breathing irregularities.

Diaphragmatic pacing starts at given intervals. In one embodiment the interval time is initially about 10 seconds. The interval is slowly increased from 11 seconds to about 15 seconds. If the patient does not breath on their own, the pacing begins again at 10-second intervals and this is repeated. If the patient begins breathing on their own, typically where the PO₂ and PCO₂ levels are normalized and the brain resumes sending nerve stimulation. The system then returns to the mode where it is sensing respiratory effort.

The methods and devices of the subject invention can also be used in conjunction with treatment methods for disorders of the autonomic nervous system, and more specifically the treatment of conditions through electrical modulation and/or pharmacological modulation of the autonomic nervous system, as described in U.S. Pat. No. 7,149,574, and published U.S. patent applications 2005/0021092 and 2005/0240241, incorporated herein by reference. The subject invention can be used in the treatment of sleep apnea and other conditions that modulate carbon dioxide levels in circulating blood and/or decrease oxygen levels in circulating blood and/or increase acidity in bodily fluids, i.e., any condition having a manifestation of hypoxia and/or hypercarbia and/or acidosis and/or hypercarbia. In addition to the embodiments described above, embodiments of the subject invention may include modulating (electrically and/or pharmacologically) at least a portion of the ANS to decrease pCO2 concentration in circulating blood, and/or increase pO2 concentration in circulating blood, and/or decrease pH in bodily fluids, and/or treat a condition having a manifestation of hypoxia and/or hypercarbia and/or acidosis, and/or hypercarbia and/or treat a condition that has resulted or is associated from any of the above (e.g., associated inflammatory conditions).

For example, conditions having a manifestation of chronic or acute hypoxia and/or hypercapnia and/or hypercarbia and/or acidosis, such as for example obstructive sleep apnea (“OSA”) and other chronic conditions described herein which include, but are not limited to, conditions that disturb or alter circulating blood concentrations of pO2, pCO2 and pH, such as chronic obstructive pulmonary disease (“COPD”), primary pulmonary hypertension (“PPHTN”), secondary pulmonary hypertension (“SPHTN”) and the like, may be treated in accordance with the subject invention, as well as the manifested circulating blood concentrations of pO2, pCO2 and pH. As noted above, treatment according to the subject invention may be continuous, intermittent or cyclical. Certain embodiments include performing a treatment protocol according to the subject invention at night time, e.g., while a subject is sleeping. For example, a pharmacological agent may be administered prior to a subject's bedtime and/or electrical modulation may be actuated during the time a subject is sleeping. Embodiments are further described primarily with reference to OSA as a condition having a manifestation of chronic or acute hypoxia and/or hypercapnia and/or hypercarbia and/or acidosis for exemplary purposes only and is in no way intended to limit the scope of the invention. It is to be understood that any condition having a manifestation of chronic or acute hypoxia and/or hypercapnia and/or hypercarbia and/or acidosis also contemplated.

The actual nerves and/or area(s) of the nerves that can be electrically stimulated or inhibited will vary in accordance with the disease or symptom being treated, and includes pre- and post ganglionic nerve fibers, as well as ganglionic structures, efferent and afferent nerve fibers, synapses, nerve plexi, etc., and combinations thereof in certain embodiments. In certain embodiments, a given nerve fiber may be electrically stimulated or inhibited in more than one area of the nerve fiber.

Targeted area(s) of the parasympathetic nervous system which can be electrically stimulated or inhibited in accordance with the subject invention include but are not limited to: the oculomotor nerve; facial nerve; glossopharyngeal nerve; hypoglossal nerve; trigeminal nerve, vagus nerve including the recurrent laryngeal branches of the vagus nerve, the pharyngeal and superior laryngeal branches of the vagus nerve, the cardiac branches of the vagus nerve, the anterior vagal trunk and the posterior vagal trunk; ciliary ganglion; pterygophalatine ganglion; vidian nerve, pterygopalatine nerve, otic ganglion; chorda tympsubmandibular ganglion; lingual nerve; submandibular ganglion; esophageal plexus; parasympathetic branch from inferior hypogastric plexus to descending colon; rectal plexus and pelvic planchnic nerves, or a combination of two or more of the above. For example, in certain embodiments electrical stimulation to the vagus, including vagal afferent nerves, may be employed.

Targeted area(s) of the sympathetic nervous system that can be electrically stimulated or inhibited in accordance with the subject invention include but are not limited to: internal carotid nerve and plexus, middle and superior cervical sympathetic ganglion; vertebral ganglion; cervicothoracic ganglion; sympathetic trunk; cervical cardiac nerves; cardiac plexus; thoracic aortic plexus; celiac ganglion; celiac trunk and plexus; superior mesenteric ganglion; superior mesenteric artery and plexus; intermesenteric plexus; inferior mesenteric ganglion; inferior mesenteric artery and plexus; superior hypogastric plexus; hypogastric nerves; vesical plexus; thoracic cardiac nerves; sympathetic trunk; 6th thoracic sympathetic ganglion; gray and white rami communicantes; greater, lesser and least splanchnic nerves; aorticorenal ganglion; lumbar splanchnic nerves; gray rami communicantes and sacral splanchnic nerves; or a combination of two or more of the above.

During the period of time that a given area of a nerve is electrically stimulated or inhibited, the electrical stimulation or inhibition may be substantially continuous, including continuous or intermittent (i.e., pulsed or periodic), where in many embodiments the electrical stimulation or inhibition is in the form of electrical pulses. In other words, in certain embodiments a given area of the parasympathetic nervous system, for example, (e.g., a given nerve fiber) may be continuously electrically stimulated during the above-described period of time and in certain other embodiments a given area of the parasympathetic nervous system (e.g., a given nerve fiber) may be pulsed or intermittently electrically stimulated during the period of time described above. In addition to electrical stimulation or inhibition, these nerves may also be blocked with neurolytic agents, cryotherapy, or radiofrequency lesioning.

### Devices

Aspects of the invention include devices and systems, including implantable medical devices and systems. The systems of the invention include an implantable device comprising a respiratory parameter sensor, a diaphragm stimulator, and a cardiac tissue stimulator, such as an implantable pulse generator as described above, that include a control means configured to transmit and/or receive a signal. The respiratory parameter sensor may be one of a diaphragm stimulator/sensor as described above, and/or a flow measuring device (e.g. a pneumotachometer), an intrathoracic pressure sensor, or a transthoracic impedance sensor as described above.

The programmable diaphragm stimulating and sensing device 130 further includes a ROM memory coupled to the programmer 140 by way of a data bus. The ROM memory provides program instructions to the programmable diaphragm stimulating and sensing device 130. The device may be programmed to provide certain stimulation parameters such as pulse or burst morphology; frequency, pulse width, pulse amplitude, duration and a threshold or trigger to determine when to stimulate. A second RAM memory (event memory) can be provided to store sensed data sensed, e.g., by the electrodes (EMG or nerve activity), diaphragm movement sensors such as strain gauge, or the accelerometer. These signals may be processed and used as programmed to determine if and when to stimulate or provide other feedback to the patient or clinician. Also stored in RAM memory may be the sensed waveforms for a given interval, and a count of the number of events or episodes over a given time as counted by the programmer 140. The system's memory will be programmable to store: number of sleep apnea episodes per night; pacing stimulation and length of time; the systemic auto-correction (i.e., how stimulus was adjusted, e.g., in amplitude frequency phase or waveform, to reach a desired or intrinsic level response); body resumption of breathing; the number of apnea episodes with specific durations and averages and trending information; hyperventilation episodes during supine position; number of hyperventilation episodes during sleep position; number of hyperventilation episodes during vertical position; and patient information including the medications and dosages and dates of changes. These signals and information may also be compiled in the memory and downloaded telemetrically to an external device when prompted by the external device.

An external device (150 in FIG. 1) may be equipped with a personal digital assistant type device that connects to the phone line for downloading the patient specific information regarding patient's pulmonary status as well as of conditions including apnea, hypoventilation and hyperventilation, and whether the parameters are programmed correctly. The system may prompt the patient with voice, music or other audible alarms regarding compliance with medication, diet and exercise. This device may allow for remote follow-up, continuous monitoring of the patient's hemodynamic status, effectiveness of the drug regime and in particular the management of diuretics where the apnea is influenced by pulmonary edema. The patient hand held also can provide a daily update regarding the status of the device and as well as whether patients need to see the physician or change dosage of a medication, based on information compared to programmed parameters by the physician inside the implantable device. The device can also manage a patient's diuretic level in relationship to breathing frequency and character.

The information may be viewed by the clinician using a web browser anywhere in the world of the handheld can send a fax or notice to the physician's office once the parameters of interest are outside the programmed range. The physician may then request an office visit. The system also can send a summarized report on weekly, biweekly, or monthly as routine update based on the decision of the physician programmed in the handheld device. Medication adjustment/drug titration may be accomplished remotely. Hand-held communication protocol/technology may be magnetic or RF.

Also provided are methods of using the systems of the invention. The methods of the invention generally include: providing a system of the invention, e.g., as described above, that includes an implantable pulse generator and an implantable diaphragm stimulation device; and transmitting a signal between the first and second devices. The system can additionally include a flow sensor, an intrathoracic pressure sensor, or a device for measuring impedance. In certain embodiments, the transmitting step includes sending a signal from the first to said second device. In certain embodiments, the transmitting step includes sending a signal from the second device to said first device. The signal may transmitted in any convenient frequency, where in certain embodiments the frequency ranges from about 400 to about 405 MHz. The nature of the signal may vary greatly, and may include one or more data obtained from the patient, data obtained from the implanted device on device function, control information for the implanted device, power, etc.

### Applications

The methods and devices of the invention, such as those described above, find use in any application where it is desired to produce RSA in a subject. In some embodiments the system is used to treat subjects with a heart failure condition, such as an early heart failure condition. Coordinating patient respiration with pacing of the heart to restore respiratory sinus arrhythmia for a period of time (e.g. while the patient is sleeping) may be therapeutic to heart failure patients by either reducing the load on the heart and/or increasing oxygen saturation levels. In one embodiment, breathing can be stimulated to increase oxygen saturation levels for a period of time, which can allow positive remodeling of the heart by reducing the load on the heart for a period of time, e.g., for one or more time intervals during sleep. The oxygen saturation levels can be increased by increasing minute ventilation and by restoring respiratory sinus arrhythmia, and subsequent reduced cardiac output for a period of time can provide an opportunity for an overloaded heart to rest. One aspect of the invention is therefore a device and method for treating a subject for a heart failure condition by providing breathing stimulation for periods of time that increase oxygen saturation levels. Examples of such breathing therapies and therapy devices are described in U.S. patent application 2005/0085868 incorporated in its entirety herein by reference. The methods of the subject invention can also be used to provide a cardiac treatment system which monitors a patient's cardiac condition over a predetermined time period and, based on his breathing pattern, indicates whether the patient's condition has improved, worsened or remaining unchanged, as described above.

In some embodiments the system is used to treat pulmonary edema, a condition often found in heart failure patients in which there is excess fluid in the lungs. When heart failure patients are lying flat, they can have central fluid accumulation and pulmonary congestion, which stimulates vagal irritant receptors in the lungs to cause reflex hyperventilation. When hyperventilation causes the PCO₂ level to fall below the threshold level required to stimulate breathing, the central drive to respiratory muscles and airflow diminishes significantly or ceases, and apnea (lack of, or attenuated breathing) ensues until the PCO₂ rises again above the threshold required to stimulate ventilation. This often results in spontaneous arousal from sleep. In some embodiments the present invention provides a method and apparatus for detecting apnea (e.g., by a lack of EMG for a given period of time), by sensing the respiratory parameter of the diaphragm and providing stimulation to the diaphragm to elicit diaphragm movement to cause respiration when respiration ceases or falls below a threshold level.

In some embodiments the system is used to treat hypertension, a condition that often co-exists in heart failure patients. Some studies have shown that patients who are coached to breathe at about 6 breaths per minute have a reduction in blood pressure, and improve resting oxygen saturation. In some embodiments, electrically stimulating the cardiac tissue and diaphragm tissue may occur only when the subject is sleeping, in order to provide the desired therapy.

In some embodiments the system is used to achieve increases and decreases in minute ventilation or other related parameters to treat breathing disorders, such as COPD (Chronic Obstructive Pulmonary Disease). COPD patients typically retain high levels of CO₂ in their blood because of difficulties in exhaling CO₂. The system can be used to manipulate PCO₂ or SaO₂ levels by controlling minute ventilation or related respiration parameters that affect minute ventilation, and low levels of inspiration with high levels of exhalation may be induced by inducing longer periods of exhalation, in order to lower the level of CO₂.

In some embodiments the system is used to treat breathing disorders, including apnea (primarily central sleep apnea), periodic breathing, and Cheyne-Stokes respiration. Cheyne-Stokes respiration and apnea tend to occur in repeated cycles in heart failure patients. This is believed to occur in part due to the delay in the feedback or chemoreceptor sensing due to circulatory delay which is common in heart failure patients. The purpose of the apnea therapy described herein is to stabilize the blood gas levels more gradually and to reduce the extreme fluctuations between Cheyne-Stokes hyperventilation and apnea. Some studies indicate that Cheyne-Stokes respiration may occur because of a drop in SaO₂ levels due to circulatory delay in heart failure patients. Apnea, especially central apnea, may be caused in part by a drop in partial pressure of CO₂ that follows a Cheyne-Stokes episode. Some studies indicate that stimulated breathing prior to or during apnea may stabilize the broad swings of blood gas concentrations that occur during cycles of Cheyne-Stokes respiration and apnea. Blood gas levels may be manipulated to prevent breathing disorders by stimulating the diaphragm after detecting a breathing disorder (see patent publication 2005/0085865, the disclosure of which is herein incorporated by reference). Furthermore, stimulation of the diaphragm in these patients may help maintain vagal tone, which is associated with restful sleeping. As mentioned above, diaphragm stimulation can also prevent a fall in O₂ saturation that would normally initiate an arousal episode during apnea, and therefore can provide a device and method for greater periods of restful sleep in patients with apnea.

Furthermore, disordered breathing may contribute to a number of adverse cardiovascular outcomes such as hypertension, stroke, congestive heart failure, and myocardial infarction. Sleep-related breathing disorders, especially central sleep apnea, have been found to have a relatively high prevalence in patients with heart failure and may have a causative or influencing effect on heart failure. In about 50% of patients with stable congestive heart failure, there is an associated sleep disordered breathing, predominantly central sleep apnea with a minority having obstructive sleep apnea. Furthermore, sleep related breathing disorders are believed to be physiologically linked with heart failure. Central sleep apnea is a known risk factor for diminished life expectancy in heart failure. It is also believed that in view of this link, treatment aimed at relieving sleep related breathing disorders may improve cardiovascular outcomes in patients with heart failure.

In some embodiments the subject methods and devices are used to treat any disease or symptom in which stabilization or alteration of blood gases, manipulation of O₂ and/or CO₂ levels, manipulation of blood pressure, or reducing the load on the heart may be desirable. A “disease” is a health condition that requires treatment or attention such as disorder, illness, ailment, affliction, condition, state, problem, obstruction, malfunction, etc. A “symptom” is any physical sign that can be observed or measured objectively (e.g., vital sign such as pulse or blood pressure, lab or test result, bruising, rash, swelling, etc.) or any feeling or sensation that is subjective (e.g. pain, nausea, dizziness, anxiety, depression, etc.), reported by a subject.

Diseases or symptoms that can be treated with the subject methods include but are not limited to: infectious and parasitic diseases, inflammatory diseases, lymphoproliferative diseases, neoplasms, endocrine, nutritional, and metabolic diseases or abnormal metabolic states, immunity disorders including autoimmune disorders, diseases of the blood and blood-forming organs, mental or behavioral disorders, neurological disorders, diseases of the brain including seizure disorders, diseases of the nervous system, movement disorders, autonomic disorders, sleep disorders, diseases of the sense organs, diseases of the circulatory system, diseases of the respiratory system, diseases of the digestive system, diseases of the genitourinary system, complications of pregnancy, childbirth, and the puerperium, diseases of the skin and subcutaneous tissue, diseases of the musculoskeletal system or connective tissue, including diseases of the joints, congential anomalies, congenital deformations, or chromosomal abnormalities, diseases originating in the perinatal period, injury, poisoning, treatment of pain, or ill-defined diseases or disorders not elsewhere classified.

### Kits

Also provided are kits that include at least one of a respiratory parameter sensor, a diaphragm stimulator, a cardiac tissue stimulator, and a control means configured to operate the device, as part of one or more components of an implantable device or system, such as the devices and systems reviewed above. In certain of these embodiments, the structure and control unit may be electrically coupled by an elongated conductive member. The respiratory parameter sensor in the kit may be one or more of a diaphragm sensor, an intrathoracic pressure sensor, or a transthoracic impedance sensor,

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of producing respiratory sinus arrhythmia in a subject, said method comprising:
electrically stimulating:
(a) cardiac tissue in said subject; and
(b) diaphragm tissue in said subject;
in a manner effective to produce respiratory sinus arrhythmia in said subject.

2. The method according to claim 1, wherein said method is a method of treating said subject for a heart failure condition.

3. The method according to claim 1, wherein said heart failure condition is an early heart failure condition.

4. The method according to claim 1, wherein said electrically stimulating occurs only when said subject is sleeping.

5. The method according to claim 1, wherein said electrically stimulating cardiac tissue is to one of a vagus nerve and sympathetic nerve system to control the beat rate of a subject's heart.

6. The method according to claim 1, wherein said electrically stimulating diaphragm tissue occurs by stimulating the phrenic nerve.

7. The method according to claim 1, wherein said method further comprises deriving a respiratory parameter in said subject.

8. The method according to claim 7, wherein said respiratory parameter is derived from diaphragm movement.

9. The method according to claim 7, wherein said respiratory parameter is derived from diaphragm movement detected by a sensing a diaphragm EMG.

10. The method according to claim 7, wherein said respiratory parameter is derived from diaphragm movement detected by a movement detector.

11. The method according to claim 7, wherein said respiratory parameter is respiratory flow.

12. The method according to claim 7, wherein said respiratory parameter is determined by sensing phrenic nerve activity.

13. The method according to claim 7, wherein said respiratory parameter is determined using a measure of intrathoracic pressure.

14. The method according to claim 7, wherein said respiratory parameter is determined using a measure of transthoracic impedance.

15. The method according to claim 1, wherein said subject is a mammalian subject.

16. The method according to claim 15, wherein said mammalian subject is a human.

17. The method according to claim 2, wherein said method further comprises diagnosing said subject for said heart failure condition.

18. A method of producing respiratory sinus arrhythmia in a subject, said method comprising:
sensing a respiratory parameter in said subject; and
electrically stimulating cardiac tissue in said subject based on said sensed respiratory parameter in said subject in a manner effective to produce respiratory sinus arrhythmia in said subject.

19. The method according to claim 18, wherein said respiratory parameter is diaphragm movement.

20-21. (canceled)

22. The method according to claim 19, wherein said respiratory parameter is respiratory flow.

23-25. (canceled)

26. The method according to claim 18, wherein said method further comprises electrically stimulating diaphragm tissue in said subject.

27. The method according to claim 18, wherein said method is a method of treating said subject for a heart failure condition.

28. The method according to claim 27, wherein said heart failure condition is an early heart failure condition.

29. The method according to claim 28, wherein said method further comprises diagnosing said subject for said heart failure condition.

30. The method according to claim 18, wherein said electrically stimulating occurs only when said subject is sleeping.

31-33. (canceled)

34. An implantable device, comprising:
at least one of a respiratory parameter sensor and a diaphragm stimulator;
a cardiac tissue stimulator; and
control means configured to operate said device in a manner sufficient to perform a method according to claim 1 or claim 18.

35-47. (canceled)
